(19) United States
(12) Patent Application Publication
(10) Pub. No.: US 2026/0043059 A1
(43) Pub. Date: Feb. 12, 2026

(54) PRODUCTION OF COLOURANTS IN ENGINEERED BIOLOGICAL SYSTEMS AND METHODS THEREON

(71) Applicant: Lite-1: Microbial Colour Ltd., Vancouver (CA)

(72) Inventors: Steven Slater, Vancouver (CA); Sarah Elizabeth Graham, Vancouver (CA); Amin Shahsavarani, Vancouver (CA)

(21) Appl. No.: 19/296,458

(22) Filed: Aug. 11, 2025

Related U.S. Application Data
(60) Provisional application No. 63/681,334, filed on Aug. 9, 2024.

(51) Int. Cl. C12P 17/16; C12N 9/02; C12N 9/88; C12P 13/22

(52) U.S. Cl. CPC C12P 17/165; C12N 9/0071; C12N 9/88; C12P 13/225; C12P 13/227; C12Y 114/18001; C12Y 401/99001

(57) ABSTRACT

Disclosed are processes and methods for production of pigment compounds, such as melanin, using engineered microbial systems and chemical modifications to intermediate compounds. One process involves producing a first intermediate (e.g., an amino acid like tyrosine) and a second intermediate (e.g., an enzyme like tyrosinase) within a microbial cell, exporting the intermediates to an extracellular medium, and enabling their reaction outside the cell to form the pigment compound. The microbial cell is genetically engineered to enhance production and export of intermediates, prevent reuptake, and optimize enzyme activity post-export. Additional intermediates or capping agents are introduced to modulate pigment properties, such as color, molecular weight, and solubility. The process may be applied to produce various pigments, including pheomelanin and violacein, and is scalable for industrial applications in textiles, cosmetics, and carbon sequestration. The system minimizes cellular toxicity, simplifies purification, and allows for tailored pigment production.

### TECHNICAL FIELD

The present disclosure generally relates to processes for production of colourants, and more particularly related to processes for extracellular production of pigment molecules in engineered biological systems.

### BACKGROUND OF THE INVENTION

Colourant polymers, including but not limited to melanin, carotenoids, flavins, quinines, monascins, violacein, prodigision, and their related polymers, are produced naturally by a wide variety of organisms. These colourant polymers serve diverse biological functions, such as providing protection against ultraviolet radiation, oxidative stress, or microbial attack, and contributing to coloration in tissues, structures, or secretions. For example, in humans, certain colourant polymers protect skin from UV radiation while in microorganisms such as Streptomyces, colorant polymers are produced as metabolic byproducts or as a result of enzymatic activity, such as that of tyrosinase or other oxidoreductases, which may degrade phenolics released by other organisms. In birds, various melanins contribute to feather colour. Melanin accumulation in soil is an important mode for sequestration of carbon, and melanin has been identified as a potential molecule for increasing the amount of carbon stored in soils. The black, brown, buff and Tyndall-blue pigments found in feathers, hairs, eyes, insect cuticle, fruit and seeds are usually melanins and are assumed to result from the action of tyrosinase.

Many have tried various genetic engineering approaches to produce colourant polymers such as melanin, whereas others have attempted to produce the colourant polymers by using naturally occurring pigment-producing microorganisms such as bacteria. But improvements in yield/hour are desirable if production of such colourant polymers is to be economical for commercial use.

In the case of melanin, previous work to produce melanin, or precursors thereof such as tyrosine, have used native strains, or engineered strains in various ways to overproduce tyrosine and then converted to melanin by co-expressing tyrosinase. In all existing cases which exploit strain engineering to produce melanin, the melanin (or precursor such as tyrosine) is synthesized inside the cell. However, many wild-type strains, such as Bacillus and Streptomyces, produce Melanin outside the cell.

The colour of the melanin is determined by the types of monomers in the random melanin copolymer. For example, melanin produced from 5,6-dihydroxyindole-carboxylic acid (DHICA) is brown, whereas melanin produced from 5,6-dihydroxyindole (DHI) is black. Dopachrome tautomerase, also known as DOPAchrome isomerase or tyrosine related protein 2 (TRP-2), converts DOPAchrome into DHICA. The same transformation is catalyzed by a different enzyme in insects and cuttlefish. DHICA is converted to indole-5,6,quinone-carboxylic acid by DHICA oxidase, also know as tyrosine related protein 1 (TRP-1). Thus, the colour of the melanin produced may be manipulated from black to brown by provision or removal of the enzymes.

In addition to eumelanin (black to brown), another important class of melanin is pheomelanin, which exhibits a red-to-yellow colour and is of significant commercial interest for use as a pigment in fabrics, foods, and cosmetics. Pheomelanin is biosynthesized when dopaquinone, an intermediate in the melanin pathway, reacts with cysteine or glutathione to form cysteinyl DOPA, ultimately leading to the formation of pheomelanin polymers. Industrial production of pheomelanin is particularly challenging due to the tendency for concurrent formation of eumelanin, which may obscure the desired colour properties of pheomelanin.

Other microbial pigment polymers beyond melanin follow similar biosynthetic patterns. For example, violacein, produced by bacteria such as Janthinobacterium and Chromobacterium, is formed through internal production of tryptophan as a precursor molecule, which may undergo a series of oxidation, non-enzymatic, and enzymatic transformations to yield violacein (generally blue-purple), indigoidine (generally blue), and/or deoxyviolacein (generally purple). Many other microbial-based colourants, such as carotenoids, flavins, and polyphenols, are also synthesized via the production of precursor molecules that are subsequently transformed into polymeric or oligomeric pigment compounds through enzymatic and chemical reactions.

Traditional metabolic engineering has often focused on the rational design of metabolic pathways, relying on extensive a priori knowledge of cellular mechanisms in order to redirect metabolite flow, revise metabolic regulation, or introduce new pathways to achieve a particular phenotype. In recent years, however, considerable advances in molecular biology and the growing availability of annotated genome sequences have made combinatorial methods of metabolic engineering an increasingly attractive approach for strain improvement.

In this disclosure, procedures for extracellular production of pigment molecules (such as melanin, carotenoids, flavins, quinines, monascins, violacein and prodigision) in engineered biological systems, are presented.

### SUMMARY

A process for extracellular production of a pigment compound using a first and second intermediates to produce the pigment compound is provided. The process includes production of the first intermediate by an engineered cell and exporting the first intermediate to an extracellular medium outside the cell, production of the second intermediate by the cell and exporting the second intermediate to the extracellular medium, the second intermediate being active upon export to the extracellular medium, and the first and second intermediates reacting at the extracellular medium to produce the pigment compound.

The first intermediate may be an amino acid, and the second intermediate may be an enzyme.

The second intermediate may be poorly active or inactive within the cell and more active after export.

The cell may be genetically engineered to overexpress one or more genes involved in the biosynthesis or export of one or more of the first and the second intermediates.

The cell may be engineered to disrupt, delete, or downregulate one or more genes encoding importers of the first intermediate to prevent reuptake of the first intermediate.

The cell may be engineered to overexpress or upregulate one or more genes encoding exporters of the first intermediate to enhance export of the first intermediate to the extracellular medium.

The second intermediate may be fused to a signal peptide that facilitates export to the extracellular medium.

The pigment compound may be a melanin molecule, and the first intermediate may be tyrosine, and the second intermediate may be a tyrosinase.

The cell may be engineered to disrupt, delete, or downregulate first genes for importing the first intermediate and/or the second intermediate, and the cell may be engineered to overexpress or upregulate second genes for exporting the first intermediate and/or the second intermediate.

The first genes may include the tyrP and aroP genes, and the second genes may include the yddG gene.

The cell may be engineered to disrupt, delete, or downregulate the tyrP and aroP genes and to overexpress or upregulate the yddG gene.

The process may further include producing a third intermediate by the cell and exporting the third intermediate outside the cell, and the third intermediate may react with the first and the second intermediates to produce the pigment compound.

The third intermediate may include an enzyme or a compound capable of altering or modulating the composition, colour, or properties of the pigment compound by reacting with the first and/or second intermediates.

The third intermediate may be selected from the group consisting of DOPAchrome tautomerase, DHICA oxidase, cysteine, and glutathione.

The cell may be engineered to overproduce and export cysteine and/or glutathione as the third intermediate to modulate the composition or colour of the pigment compound.

The cell may be engineered to overexpress a cysteine exporter selected from the group consisting of YdeD, Bcr, TolC, and CydDC.

The cell may be engineered to express a feedback-insensitive serine O-acetyltransferase to increase cysteine biosynthesis.

The cell may be engineered to disrupt, delete, or downregulate one or more genes encoding cysteine importers.

The cell may be engineered to modulate the expression of tyrosinase to control the rate of dopaquinone formation.

The process may further include growing the cell in a growth medium.

The growth medium may include a fermentation medium or fermentation broth, and the process may be performed by batch, fed-batch, or continuous fermentation.

The cell may be grown anaerobically to minimize polymerization of the pigment compound and to accumulate intermediates of the pigment compound.

The intermediates may be separated from a biomass, the biomass resulting from the culture growth, for later use in other chemical processes, including production of the pigment compound.

The intermediates capable of polymerization into the pigment compound may be added to the growth medium to generate desired pigment polymers.

The process may further include adding one or more terminating agents to the extracellular medium to modulate the molecular weight, solubility, or color of the pigment compound.

The terminating agent may be selected from the group consisting of thiol-containing compounds, N-acetylcysteine, mercaptosuccinic acid, thioglycolic acid, resorcinol, and tyramine.

The terminating agent may be added during fermentation and/or after fermentation.

An amount and timing of addition of the terminating agent may be selected to achieve a desired average molecular weight or solubility of the pigment compound.

A pigment composition produced by some of the foregoing processes is provided, the pigment having a controlled molecular weight and being soluble in aqueous solution due to the presence of capping or terminating groups.

The cell may be capable of replicating in under 4 hours.

The cell may be capable of replicating in under two hours.

The cell may be capable of replicating in under 1 hour.

The cell may be capable of replicating in under 30 minutes.

The processes may further include introducing the cell into an outdoor environment, including soil, roots, seeds, or plants. The cell may produce and/or facilitate the accumulation of pigment compounds that increase the concentration of fixed carbon in the environment.

The pigment compound may be a violacein molecule, the first intermediate may be tryptophan, and the second intermediate may be a tryptophanase.

An engineered microbial cell for improved production of a pigment compound relative to a parental strain is provided. The pigment compound may be produced outside the microbial cell by reacting a first intermediate and a second intermediate to produce the pigment compound. The microbial cell is configured to produce the first intermediate and exporting the first intermediate outside the cell, produce the second intermediate and exporting the second intermediate outside the cell, the second intermediate being poorly active or inactive within the cell and more active after export.

A process for modulating growth of a colourant polymer is provided, the process including adding a chain terminating molecule to a polymerization reaction of the colourant polymer, the chain terminating molecule reacting with polymer extension sites of the growing colourant polymer, oligomers thereof, or monomers thereof, and thereby blocking further extension of the colourant polymer.

The colourant polymer may be produced by polymerization of one or more reactive colourant intermediates of the colourant polymer, and the one or more colourant intermediates may be produced or derived from intermediate compounds produced by an engineered microbial cell.

The microbial cell may be genetically engineered to overproduce and export the one or more colourant intermediates into an extracellular medium.

The colourant polymer may be melanin polymer selected from the group consisting of eumelanin, pheomelanin, neuromelanin, allomelanin, pyomelanin, and a related polymeric molecule, and the reactive colourant intermediate may include one or more of dopaquinone, 5,6-dihydroxyindole, 5,6-dihydroxyindole-2-carboxylic acid, or mixtures thereof.

The terminating agent may be a thiol-containing compound.

The thiol-containing compound may be selected from the group consisting of N-acetylcysteine, mercaptosuccinic acid, thioglycolic acid, cysteine, and glutathione.

The terminating agent may include a phenolic or aromatic amine compound selected from the group consisting of resorcinol, and tyramine.

The amount and timing of addition of the terminating agent may be selected to achieve a target average molecular weight, solubility, or colour of the colourant polymer.

The process may further include growing the microbial cell in a growth medium including a fermentation medium or fermentation broth, and the process may be performed by batch, fed-batch, or continuous fermentation, and the chain terminating molecule may be added during fermentation and/or after fermentation.

A process for extracellular production of a colourant compound is provided, the process including producing a first intermediate by an engineered microbial cell and exporting the first intermediate to an extracellular medium, producing a second intermediate by the microbial cell and exporting the second intermediate to the extracellular medium, the second intermediate being active upon export, and reacting the first and second intermediates in the extracellular medium to produce the colourant compound.

The first intermediate may be an amino acid, and the second intermediate may be an enzyme.

The second intermediate may be poorly active or inactive within the cell and more active after export.

The cell may be engineered to overexpress or upregulate one or more genes encoding exporters of the first intermediate, and to disrupt, delete, or downregulate one or more genes encoding importers of the first intermediate to prevent reuptake of the first intermediate.

The second intermediate may be fused to a signal peptide that facilitates export to the extracellular medium.

The colourant compound may be a melanin molecule, the first intermediate may be tyrosine, and the second intermediate may be a tyrosinase.

The colourant compound may be a violacein molecule, the first intermediate may be tryptophan, and the second intermediate may be a tryptophanase.

The cell may be engineered to disrupt, delete, or downregulate the tyrP and aroP genes and to overexpress or upregulate the yddG gene.

The process may further include producing a third intermediate by the cell and exporting the third intermediate outside the cell, and the third intermediate may react with the first and the second intermediates to produce the colourant compound.

The third intermediate may include an enzyme or a compound capable of altering or modulating the composition, color, or properties of the colourant compound by reacting with the first and/or second intermediates.

The third intermediate may be selected from the group consisting of DOPAchrome tautomerase, DHICA oxidase, cysteine, and glutathione.

The cell may be engineered to overproduce and export cysteine and/or glutathione as the third intermediate by overexpressing a cysteine exporter and disrupting, deleting, or downregulating one or more genes encoding cysteine importers.

The process may further include cultivating the cell in a growth medium, a fermentation medium, or broth, and the process may be performed by batch, fed-batch, or continuous fermentation.

The cultivation or fermentation may be conducted under anaerobic conditions to minimize polymerization of the colourant compound and to accumulate intermediates of the colourant compound.

The intermediates may be separated from a biomass resulting from the culture growth for later use in other chemical processes including production of the colourant compound.

The process may further include adding one or more terminating agents to the extracellular medium to modulate the molecular weight, solubility, or color of the colourant compound.

The one or more terminating agents may be selected from the group consisting of thiol-containing compounds, phenolic compounds, and aromatic amines.

The one or more terminating agents may be added during fermentation and/or after fermentation, and the amount and timing of the addition of the one or more terminating agents may be selected to achieve a desired average molecular weight or solubility of the colourant compound such that the resulting colourant compound may have a controlled molecular weight and may be soluble in aqueous solution.

The processes may further include introducing the microbial cell into an outdoor environment, including soil, roots, seeds, or plants, and the microbial cell may produce and/or may facilitate the accumulation of pigment compounds that increase the concentration of fixed carbon in the environment.

A genetically engineered microbial cell for improved production of a pigment compound relative to a parental strain is provided. The pigment compound is produced outside the microbial cell by reaction of a first intermediate and a second intermediate, the microbial cell configured to produce the first intermediate and export the produced first intermediate outside the cell and produce the second intermediate and export the produced second intermediate outside the cell, the second intermediate being poorly active or inactive within the cell and more active after export.

Other aspects and features will become apparent, to those ordinarily skilled in the art, upon review of the following description of some exemplary embodiments.

### BRIEF DESCRIPTION OF THE DRAWINGS

In the following, embodiments of the present disclosure will be described with reference to the appended drawings. However, various embodiments of the present disclosure are not limited to the arrangements shown in the drawings.

FIG. 1 is a diagram showing the biochemical transitions leading from tyrosine to various melanin intermediates to form melanin copolymers. The diagram also shows enzymes that catalyze certain reactions;

FIG. 2 is a diagram showing biosynthesis of melanin extracellularly using an engineered E. coli;

FIGS. 3A and 3B are schematic diagrams showing extracellular production of generic pigment molecules using engineered microorganisms;

FIG. 4 is a process flow diagram outlining the elements for extracellular production and biosynthesis of a pigment compound using engineered microbial systems; and

FIG. 5 is a schematic diagram showing a schematic capping agent configured to react with reactive sites of melanin polymer or monomers or oligomers thereof.

### DETAILED DESCRIPTION

Various compositions or processes will be described below to provide an example of each claimed embodiment. No embodiment described below limits any claimed embodiment and any claimed embodiment may cover processes or compositions that differ from those described below. The claimed embodiments are not limited to compositions or processes having all the features of any one composition or process described below or to features common to multiple or all of the compositions described below.

The present disclosure relates to biosynthesis of a pigment extracellularly by producing precursors of the pigment inside a biological cell and then transporting the precursors outside the cell to react and produce the pigment extracellularly. In one example of this disclosure biosynthetic production of melanins is presented. Improved production of melanins may be achieved by genetically engineering microorganisms. The present disclosure includes: (a) genes, promoters, signal sequences and regulatory sequences capable of transforming microorganisms to produce microorganisms with the capability for enhanced production of melanin, the precursors and derivatives; (b) microorganisms with the capability for enhanced production of melanins; and (c) a process of producing melanins with a microorganism.

In particular, the present disclosure provides methods and engineered biological systems for the extracellular production of variations of melanins including eumelanins and pheomelanins by controlling the ratio of two key reactants: cysteine (or glutathione) and dopaquinone (derived from tyrosine).

The following definitions are used throughout the presented disclosure.

Melanin: Melanins are polymers formed by the polymerization of reactive intermediates through mechanisms such as autoxidation, enzyme-catalyzed oxidation, and free radical-initiated polymerization. The reactive intermediates are chemically or enzymatically derived from precursors. Suitable enzymes include peroxidase, catalases, polyphenol oxidases, tyrosinases, tyrosine hydroxylases, and laccases. The precursors, hydroxylated aromatic compounds, include but are not limited to phenols, polyphenols, aminophenols, and thiophenols of aromatic or polycyclic aromatic hydrocarbons, such as phenol, tyrosine, pyrogallol, 3-aminotyrosine, thiophenol, and α-naphthol; as well as aromatic heterocyclic or heteropolycyclic hydrocarbons like 2-hydroxypyrrole, 4-hydroxy-1,2-pyrazole, 4-hydroxypyridine, 8-hydroxyquinoline, and 4,5-dihydroxybenzothiazole.

Pheomelanin: Pheomelanin is a type of melanin polymer characterized by its red-to-yellow colour, formed when DOPAquinone reacts with cysteine or glutathione to yield cysteinyl DOPA and subsequent sulfur-containing intermediates. The presence and relative abundance of pheomelanin versus eumelanin in the final polymer determines the visible colour of the pigment.

Tyrosine: Tyrosine is an aromatic amino acid among 20 standard amino acids forming proteins, which has been widely used in food, feed, medicine and other fields. L-tyrosine is a metabolite found in or produced by Escherichia coli (e.g., strain K12, MG1655) and other microbial species, as well as all other life forms. The L-tyrosine may promote synthesis of catecholamines, thyroid hormones and melanin in human bodies, which has an important effect on development and metabolism of humans and animals.

Cysteine is a sulfur-containing amino acid among 20 standard amino acids forming proteins, which has been widely used in food, feed, medicine and other fields. L-cysteine is a metabolite found in or produced by Escherichia coli (e.g., strain K12, MG1655) and other microbial species, as well as all other life forms. L-cysteine is involved in disulfide bonding in many proteins and is often involved in metal binding at enzyme active sites, and is a precursor to antioxidants, including glutathione.

Tyrosinase: Tyrosinase (monophenol, o-diphenol: oxygen oxidoreductase, EC: 1, 14, 18, 1) is an enzyme that is produced by plants, fungi, prokaryotes and mammals. Tyrosinase catalyzes phenolic compounds, including the amino acid tyrosine and therefore belongs to the class of enzymes called phenoloxidases. Tyrosinase o-hydroxylates monophenols to diphenols (monophenolase activity) and further o-oxidizes diphenols to quinones (diphenolase activity). Tyrosinase enzyme is responsible for oxidation of tyrosine, leading to the production of dihydroxyphenylalanine or DOPA, which is further converted by the enzyme to L-DOPAquinone, which then spontaneously polymerizes to melanin through a multi-level pathway.

Dopachrome Tautomerase is an enzyme that belongs to the tyrosinase family. Dopachrome Tautomerase plays a significant part in the synthesis of melanin pigments by converting DOPAchrome to carboxylated derivative DHICA, preventing the spontaneous decarboxylation of DOPAchrome.

DHICA oxidase: dihydroxyindole carboxylic acid oxidase, or 5,6-dihydroxyindole-2-carboxylic acid oxidase, is an enzyme that catalyzes the oxidation of 5,6-dihydroxyindole-2-carboxylic acid (DHICA) into indole-5,6-quinone-2-carboxylic acid which influences the type of synthesized melanin.

Colourant polymers refer to any polymer that is used to add colour to a substrate in which a change in colour from the native state is desired. Many colorant polymers, similar to melanins, are polymers formed by polymerization of reactive intermediates through mechanisms such as autoxidation, enzyme-catalyzed oxidation, and free radical-initiated polymerization, where these reactive intermediates are chemically or enzymatically derived from precursors. Colourant polymers may be referred herein as pigment polymers as well. Furthermore, in this disclosure “colourant” and “pigment” may be used interchangeably.

Colourant precursor or colourant intermediate (or precursor or intermediate in short), generally refer to a compound that serves as a substrate for further enzymatic or chemical transformation to yield a reactive pigment intermediate. Reactive colourant intermediates refer to a compound that is capable of undergoing polymerization to form a colourant polymer. Examples of intermediates include tyrosine, cysteine, and tryptophan, while examples of reactive intermediates include dopaquinone, DHICA, or DHI.

Extracellular medium refers to the culture or fermentation broth, or any liquid environment external to the microbial cell, in which secreted intermediates, pigment precursors, and enzymes accumulate and react.

Growth medium or growth media: as used herein encompasses any medium or media suitable for cultivation of microbial cells, including but not limited to fermentation media, fermentation broth, nutrient broth, minimal media, or any other liquid or solid medium in which the cells grow and/or produce the desired colourant compounds or their precursors.

Chain terminator: A molecule capable of reacting with one or more sites for extension of a polymer to physically or chemically block further extension of the polymer.

“Active” refers to the state of a molecule, such as an enzyme, in which an active site or functional domain is properly structured and chemically configured to bind specific substrate(s) and catalyze, facilitate, or participate in a particular biological, chemical, or catalytic reaction. An enzyme or protein is considered “active” if capable of performing the intended function under relevant conditions, as determined by any suitable assay or functional test, including but not limited to substrate conversion, product formation, pigment generation, or other measurable biochemical or physical changes. The term “active” encompasses full, partial, or enhanced activity relative to a reference or control, and does not use maximal or wild-type activity unless otherwise specified.

“Inactive” refers to the state of a molecule, such as an enzyme, protein, or catalyst, in which an active site or functional domain is absent, improperly configured, or otherwise unable to bind substrate(s) or catalyze, facilitate, or participate in the intended reaction under the relevant conditions. An “inactive” molecule exhibits no detectable activity in any suitable assay or functional test, such that there is no measurable product formation, substrate conversion, pigment generation, or other relevant biochemical or physical change above the background or negative control.

“Poorly active” refers to the state of a molecule, such as an enzyme, protein, or catalyst, in which an active site or functional domain is present and capable of binding substrate(s) or interacting with reactants, but exhibits a reduced level of activity compared to a reference, wild-type, or control molecule under comparable conditions. A “poorly active” molecule may catalyze, facilitate, or participate in the intended reaction at a significantly lower rate or efficiency, resulting in diminished but still detectable product formation, substrate conversion, pigment generation, or other measurable biochemical or physical changes, as determined by any suitable assay or functional test.

“Overexpression” refers to the condition in which a gene, polynucleotide, or genetic element is transcribed and/or translated at levels greater than those observed in a corresponding wild-type, unmodified, or parental cell under comparable conditions, resulting in increased amounts of corresponding RNA and/or protein product within the cell. Overexpression may be achievedin a variety of ways, including but not limited to the use of strong or inducible promoters, increased gene copy number, codon optimization, or removal of negative regulatory elements. In the context of pigment production, overexpression may relate to genes encoding enzymes, transporters, or regulatory proteins involved in the biosynthesis, export, or modification of pigment precursors, intermediates, or pigment polymers, thereby enhancing the cellular or extracellular accumulation of pigment-related molecules.

“Overproduction” refers to the condition in which a cell, organism, or system produces a molecule, such as a protein, enzyme, metabolite, pigment precursor, pigment intermediate, or colourant polymer, at levels greater than those observed in a corresponding wild-type, unmodified, or parental cell under comparable conditions. Overproduction may result from overexpression of one or more genes, metabolic engineering, pathway optimization, or any other modification or process that increases the cellular or extracellular concentration of the molecule of interest. In the context of colourant production, overproduction may relate to enhanced accumulation of pigment precursors, intermediates, or final colourant polymers, either within the cell or in the extracellular medium, thereby increasing the overall yield or availability of pigment-related compounds.

Microorganisms producing melanins are widely distributed in nature, including but not limited to Streptomyces, Rhizobium, Agaricus, Ustilago, Cryptococcus, Gluconobacter, Pseudomonas, Xanthomonas, Cochliobolus, Pleospora, Alternaria, Aurobasidium, Botrytis, Cladosporium, Diplodia, Sclerotium, Verticillium, Eurotium, Aspergillus, Stachybotrys, Hendersonula, Streptoverticillium, and Micromonospora.

Many microorganisms are genetically engineered to produce melanins, including Streptomyces, Escherichia, Bacillus, Streptococcus, Salmonella, Staphylococcus, and Vibrio.

In an embodiment, microorganisms that produce melanins or melanin analogs are enhanced through modifications such as plasmid insertion and mutation. The microorganisms may be exposed to ultraviolet or gamma radiation or treated with mutagenic chemicals to induce mutations, and the resulting cells screened or selected to produce beneficial phenotypes.

It will be appreciated that, although much of the present disclosure relates to pigment production in with E coli cells, the present disclosure is applicable to a pigment production and improving pigment production with a wide variety of microbial cells.

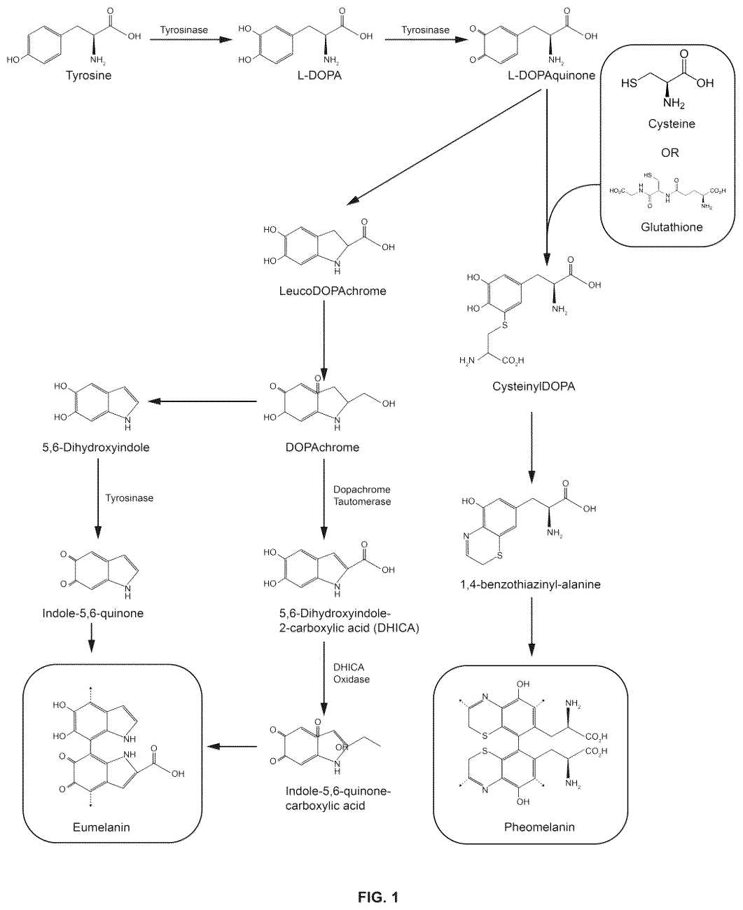

Referring to FIG. 1, several pathways for converting tyrosine to eumelanin or pheomelanin are illustrated. Various biochemical transitions are presented that lead to production of various melanin intermediates and copolymers from tyrosine. The transitions, for example, include conversion of tyrosine to L-DOPA, L-DOPA to L-DOPAquinone, L-DOPAquinone to CysteinylDOPA, CysteinylDOPA to 1,4-benzothiazinyl-alanine, and 1,4-benzothiazinyl-alanine to Pheomelanin. In FIG. 1, when an enzyme catalyzes a reaction (e.g., Dopachrome Tautomerase catalyzes DOPAchrome to DHICA), the enzyme name appears next to the appropriate transition arrow. The conversion of DOPAquinone to cysteinylDOPA may utilize either cysteine or glutathione. Spontaneous reactions have no enzyme indicated (e.g. conversion of L-DOPAquinone to LeucoDOPAchrome). Both eumelanin and pheomelanin may exist as large, random heteropolymers, and FIG. 1 only indicates one type of dimer that may exist within the polymer.

The direction of dopaquinone flow toward eumelanin or pheomelanin largely depends on the likelihood of dopaquinone encountering cysteine (or glutathione) versus another dopaquinone molecule. Thus, by selectively controlling the synthesis or presence of melanin precursors, the melanin biosynthesis may be modulated toward production of eumelanin or pheomelanin. For example, by creating a large excess of cysteine relative to tyrosine and by controlling the rate of dopaquinone formation (e.g., by limiting tyrosinase activity), the biosynthesis reactions may be biased toward the formation of pheomelanin.

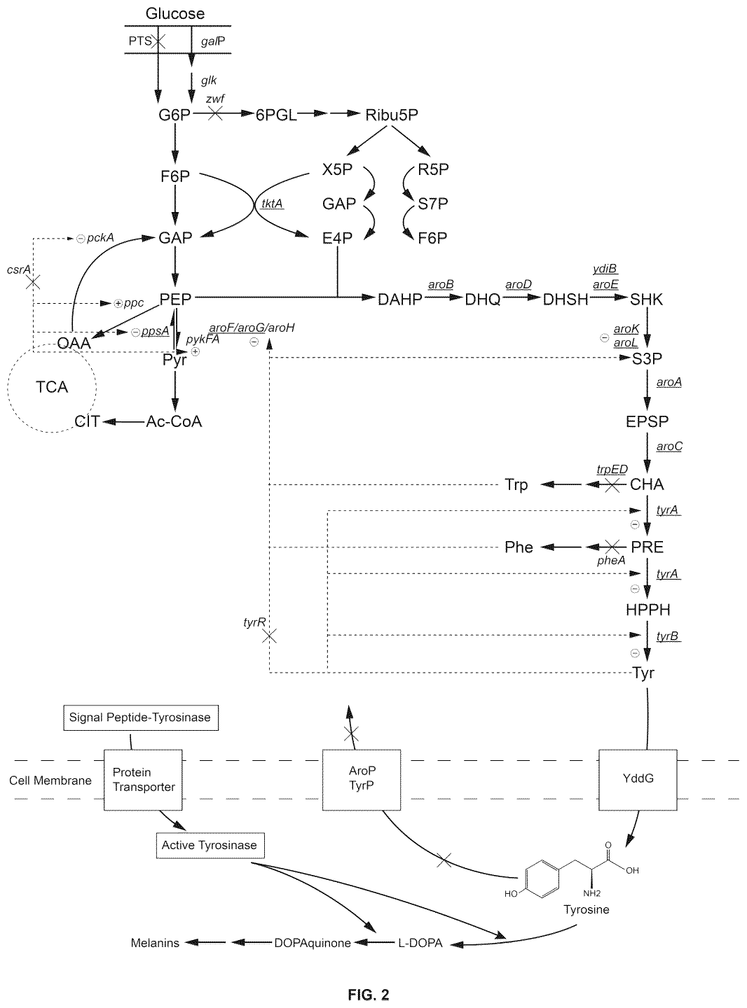

Referring to FIG. 2, an engineered E. coli bacteria modified to produce extracellular melanins is presented according to one embodiment. The engineered E. coli produces melanins by overproducing tyrosine in the cell, exporting the produced tyrosine outside of the cell while preventing net import or backflow of tyrosine to inside the cell, producing tyrosinase while the tyrosinase is minimally active inside the cell (to minimize reaction with produced tyrosine and subsequently producing melanin in vivo), exporting tyrosinase outside the cell and making the tyrosinase more active outside the cell, and finally producing melanins by reactions between the tyrosine and the tyrosinase extracellularly. FIG. 2 presents an overview of pathways from glucose to extracellular production of melanin. Chemical intermediates and key pathways are indicated by abbreviations in capital letters and are recognizable by those skilled in the art of biochemistry. Aromatic amino acids are designated by their standard 3-letter abbreviations, Tyr, Trp and Phe. Genes relevant to key processes are italicized according to standard E. coli nomenclature. Transporters in the membrane are shown within boxes. Chemical transitions are designated by arrows. Any arrow with an “X” through it indicates that the process is disrupted.

The engineered E. coli may be obtained from an E. coli as an original strain and then subjecting the strain to the following gene editing processes: disrupting a gene tyrP encoding a tyrosine and H (+) symporter, disrupting a gene aroP encoding the AroP active transporter of aromatic amino acids (e.g. tyrosine), and overexpressing an endogenous gene yddG encoding an amino acid passive transporter YddG. The original E. coli strain may be a modified strain that overproduces tyrosine intracellularly. For example, the original E. coli may be selected as the strain as according to Chávez-Béjar et.al., Biological Production of L-Tyrosine and Derived Compounds. Process Chemistry (2012) or as shown in FIG. 2. Other methods for overproduction of tyrosine may also be applied, such as that of Juminaga et al, Modular Engineering of L-Tyrosine Production in Escherichia Coli. (2012).

According to another embodiment, in order to favor the biosynthesis and production of pheomelanin, cysteine and/or glutathione are added to the extracellular media or alternatively may be synthesized in vivo of the same E. Coli cell or another cell and the exported to the extracellular media. The addition of cysteine or glutathione may produce melanin of a different colour spectrum. Analogous to the methods used to overproduce tyrosine, E. coli, or other potential microorganisms, may be engineered to overproduce and export cysteine and/or glutathione. For example, overproduction of cysteine may be accomplished by introducing genetic modifications such as expression of feedback-insensitive serine O-acetyltransferase, weakening the degradation of L-cysteine through the removal of L-cysteine desulfhydrases, and overexpression of cysteine exporters, including but not limited to ydeD, bcr, tolC, and cydDC. Although E. coli does not generally possess a high-affinity cysteine importer, although cysteine may be imported through more generalized amino acid transport systems, and thus the organism engineering system may also include deletion or down-regulation of cysteine importers to minimize reuptake of exported cysteine. In certain embodiments, alternative biosynthetic pathways are utilized depending on the microbial species, such as those initiating cysteine biosynthesis from methionine rather than serine. The engineering strategies, collectively enable elevated concentrations of cysteine in the extracellular space, thereby facilitating modulation of pigment composition and properties.

In general, bacteria are focused on importing amino acids, and they have specific proteins to do so. For example, the tyrP gene in E. coli encodes an ATP-dependent, tyrosine-specific import protein, so in normal conditions, the bacteria consumes energy to pull tyrosine into the cell. AroP is another gene that encodes an ATP-dependent aromatic amino acid (such as tyrosine) import permease. However, in the engineered E. coli bacteria shown in FIG. 2 the E. coli is engineered to disrupt tyrP and aroP genes to reduce import or backflow of tyrosine into the cell and the associated ATP consumption. According to one embodiment, the tyrP gene are constitutively derepressed by the tyrR mutation that is made to overproduce tyrosine as shown in FIG. 2. According to one embodiment, the methods described in patent of Jingwen Zhou et. al, Recombinant Escherichia Coli for Producing L-Tyrosine and Application Thereof (2024) may be used to disrupt tyrP and aroP genes in E. coli.

Additionally, in the engineered E. coli bacteria shown in FIG. 2, tyrosine flows out of the cell via the endogenous YddG protein. The YddG protein of E. coli is a passive transporter of aromatic amino acids (such as tyrosine) that facilitates net movement down their concentration gradient. In other embodiments, other passive transporters may be used to facilitate tyrosine export. Overproduction of YddG, or similar proteins, may promote flow of tyrosine out of the cell as the tyrosinase converts tyrosine to melanin, thereby creating a tyrosine gradient that favors its export. Knocking out the tyrP and aroP genes reduces the ability of the cell to actively work against this gradient. In one embodiment, a heat induced expression vector pAP-B03 plasmid is used for freely expressing the yddG gene.

In other embodiments, other genes may be knocked out or overexpressed to cause net outflow of tyrosine (or derivate metabolites) and prevent backflow to the cell.

Similarly, genes involved in cysteine import may be disrupted to further favor accumulation of cysteine in the extracellular environment, thereby increasing the probability of dopaquinone reacting with cysteine to form pheomelanin.

Furthermore, the engineered E. coli produces tyrosinase. For example, the E. coli engineering presented by Guy Della-Ciopa et al., Melanin Production by Transformed Microorganisms (2001) may be used to induce tyrosinase-producing genes (by using mel locus from plasmid vector plJ702 which includes a tyrosinase coding sequence). The induced tyrosinase-producing gene may be fused to a transport peptide to facilitate export of the produced tyrosinase outside of the cell. In some embodiments, haemolysin or YebF carriers may be used. Preferably, tyrosinase are inactive until the tyrosinase enzyme is released to the outside of the cell. Preferably, the tyrosinase does not remain in the periplasmic space, and is not active therein. Tyrosinase enzyme converts tyrosine to L-Dopa and then DOPAquinone, which then spontaneously polymerizes to form melanin.

In the context of pheomelanin production, the level of tyrosinase expression and export may be modulated to ensure that dopaquinone is produced at a rate that does not exceed the availability of cysteine, thereby favoring the formation of cysteinyl DOPA and subsequent pheomelanin.

In some embodiments, expressing enzymes that are inactive (or poorly active) within the cell and active (or more active) after export, facilitates all melanin synthesis occurring extracellularly.

The engineered E. coli in FIG. 2 may be inoculated and grown or fermented to produce high yields of melanin under suitable culture medium (or fermentation medium) under conditions of appropriate pH, temperature and oxygen, for example. According to one instance, appropriate pH is between 7-8.5 and the temperature is between 25-42 degrees Celsius. During the growth or fermentation process of the engineered E. coli cells, as tyrosine is exported and converted to melanin, the melanin are a metabolic draw. Thereby, melanin provides a strong sink, thermodynamically pulling tyrosine out of the cells via passive transport (e.g. mediated by YddG). The process may permit the cells to continue making tyrosine until, for example, they run out of carbon and/or nitrogen, or they stop metabolizing, for example, due to accumulation of waste products in the growth media. Metabolically active cells (for example, in a chemostat, fed batch, or via other manipulations), may produce melanin continuously and accumulate in or precipitate out of the growth or fermentation broth, providing continuous melanin synthesis and permitting easy purification.

In some embodiments, the engineered cell may be configured to replicate in under 4 hours. In other embodiments, the engineered cell may be configured to replicate in under 2 hours, 1 hour, or 30 minutes. In some embodiments, the engineered cell may be configured to replicate in under any time between 30 minutes to 4 hours.

The above-mentioned biosynthesis system and process for production of melanin may be generally performed by other engineered microorganisms (e.g. yeasts, bacteria, fungi) other than E. coli. As mentioned before, other microorganisms such as Streptomyces, Bacillus, Streptococcus, Salmonella, Staphylococcus, and Vibrio, may be genetically engineered to produce melanins extracellularly according to the mentioned process.

The biosynthesis system and process described under FIG. 2 may be generalized to produce other pigment molecules and compounds such as violacein, flexirubin, cartenoids, indigoidine, and riboflavin. Accordingly, other engineered microorganisms may be used to produce precursors of the target pigment compound intracellularly and then transporting them to produce the target pigment compounds extracellularly. For example, in some embodiments, a microorganism may be engineered to produce target pigment violacein by producing tryptophan (a first intermediate) and tryptophanase (a second intermediate). Other enzymes may be also produced by the engineered microorganism or from other sources to produce the target violacein pigment compound.

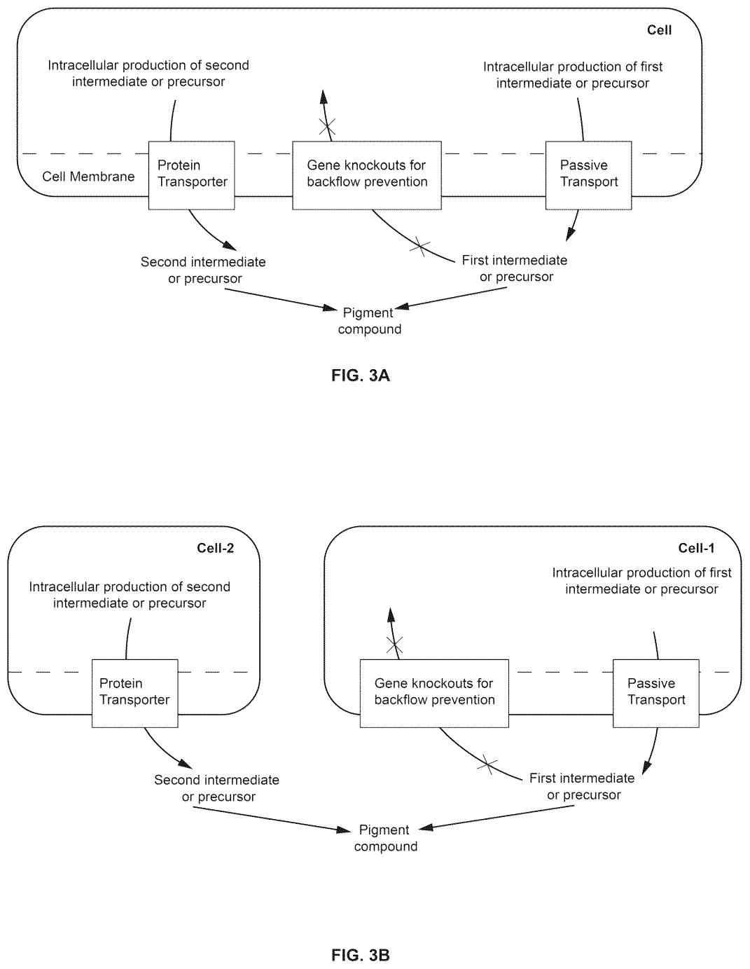

Referring to FIG. 3A, a biosynthesis of pigment compounds is generally presented according to one embodiment. A first and a second intermediary elements or precursors of the pigment molecule are produced using a single cell. The first intermediary element (e.g. tyrosine) is produced by the cell and is exported using a passive transport (e.g. using YddG), while gene disruptions (e.g. aroP and tyrP disruptions) prevent backflow of the first element to the cell. The second intermediary element (e.g. tyrosinase) is also produced inside the cell and is preferably inactivate inside the cell (e.g. due to being fused in signal peptides) while being active once transported outside the cell. The first intermediary element and the second intermediary, in active state, react to synthesize the pigment compound.

Referring to FIG. 3B. The first intermediary element is produced in a first cell (i.e. cell-1) and the second intermediary element is produced by a second cell (i.e. cell-2). For example, an E. coli as presented by No. US20240166986A1 byJingwen Zhou et al., Recombinant Escherichia coli for Producing L-Tyrosine and Application Thereof (2024) may be used to overproduce tyrosine, while tyrosinase enzyme may be overproduced by a Gliocephalotrichum fungus, or by a Streptomyces bacterium. In such embodiments, the second intermediary element may not be active while inside the cell and may be active once exported outside the cell. Preferably, neither the first nor second cell is capable of efficiently importing tyrosine.

In another embodiment, the third and/or fourth intermediate element or metabolites (e.g. DOPAchrome tautomerase and DHICA oxidase) can be supplied by the same strain producing the tyrosinase and/or tyrosine, or by a different strain(s) in the same fermentation culture.

In another embodiment the first intermediate elements or metabolites (e.g. tyrosine) are not produced by a cell in the growth culture or fermentation broth, but rather supplied as a feed into a reactor in which the engineered microorganism is producing the second intermediates and metabolites (tyrosinase). Alternatively, the second intermediate element or metabolites (e.g. tyrosinase) may be supplied as a feed into the reactor in which the engineered microorganism are producing the first intermediates and metabolites (e.g. tyrosine).

In some embodiments, cysteine and/or tyrosine are supplemented in the media at defined ratios, or chain-terminating analogs of cysteine or dopaquinone are added to control the degree of polymerization and the colour of the resulting pheomelanin.

In another embodiment the second and/or third and/or fourth intermediate element or metabolites (e.g. tyrosinase, DOPAchrome tautomerase and/or DHICA oxidase) are supplied as a feed into the reactor in which the engineered microorganism are producing the first intermediates and metabolites (e.g. tyrosine). Similarly, certain monomers, or precursors of monomers (for example, as illustrated in FIG. 1), that incorporate useful functional groups (such as a linker that may later be used to bind the dye to a fabric, or structures that change the colour of the resulting melanin), may be either produced within the cell or added to the growth medium for incorporation in the melanin polymer. The molecules may be natural or synthetic molecules.

Alternatively, since the final spontaneous stages of melanin synthesis use oxygen, metabolic intermediates to melanin, including L-DOPAquinone, leucoDOPAchrome, DOPAchrome, 5,6-dihydroxyindole (DHI), indole-5,6-quione, 5,6-dihyroxyindole-2-carboxylic acid (DHICA), and indole-5,6-quinone-carboxylic acid can be produced under anaerobic conditions and isolated from the broth under anaerobic conditions. Metablolic intermediates to melanin may later be polymerized to form melanin, or potentially other products, under controlled conditions.

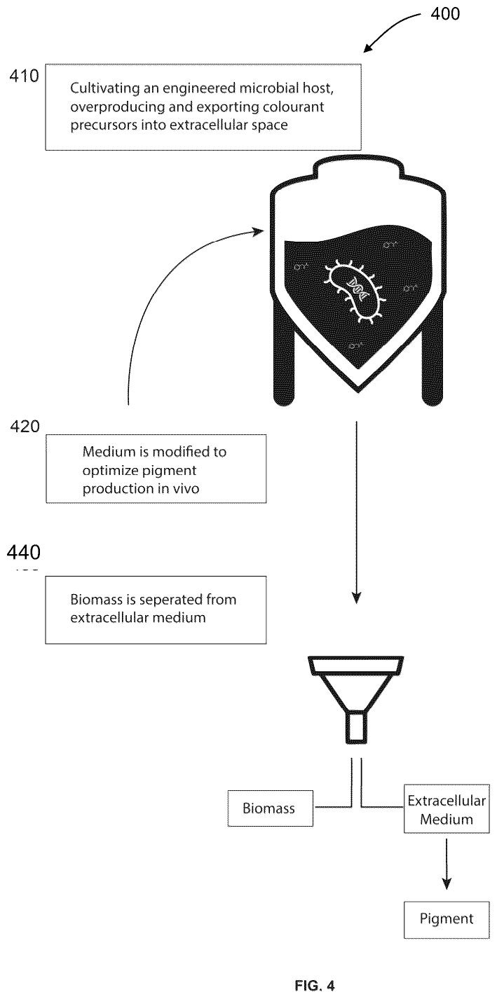

Referring to FIG. 4, a flow process for the disclosed extracellular production and biosynthesis of a pigment compound is generally presented at 400, according to one embodiment. The process 400 for microbial-based pigment biosynthesis begins with cultivating an engineered microbial host at 410, such as E. coli, to overproduce and export pigment precursors in the extracellular space. The engineered microbial cells are cultivated in a suitable growth medium or fermentation medium under controlled conditions. During cultivation or fermentation, the cells secrete the engineered pigment precursors and enzymes into the extracellular space. The cultivation or fermentation process may be performed in a bioreactor, chemostat, or fed-batch system, and the conditions such as pH (e.g., between 7 and 8.5) and temperature (e.g., between 25° C. and 42° C.) are optimized for cell growth and metabolite production. The process may be conducted under aerobic or anaerobic conditions, depending on the desired accumulation of intermediates and the minimization of premature pigment polymerization. The engineered cells secrete pigment intermediates and enzymes into the extracellular medium, resulting in an extracellular medium that grows in the secreted components and serving as the reaction environment for the final pigment or colourant biosynthesis.

The process 400, may further include elements prior to 410 to engineer a suitable microbial host (e.g., E. coli) to overproduce and export pigment precursors. Genetic modifications are introduced to enhance the biosynthetic pathways for key intermediates (e.g., tyrosine and cysteine), overexpressing specific exporters (such as YddG for tyrosine and YdeD/Bcr/TolC/CydDC for cysteine), and disrupting importers to prevent reuptake of the precursors. Additionally, the microbe is engineered to express and export pigment-synthesizing enzymes, such as tyrosinase, often fused to a signal peptide to ensure efficient secretion.

At 420, the extracellular the medium is further modified to optimize pigment production, which may include adjusting the pH, removing inhibitory byproducts, or supplementing the medium with additional precursors or modulators, such as cysteine, tyrosine, or chain-terminating agents. Theadditiona precursor or modulators allow for precise control over the reactant ratios, for directing the biosynthetic pathway toward the desired pigment, such as favoring pheomelanin over eumelanin. Additional modifications may include adjusting solubility and oxidation conditions, for example by lowering the pH to increase precursor solubility or by adding antioxidants to prevent unwanted oxidation of sensitive intermediates like cysteine. If desired, additional purified enzymes may also be introduced if desired to drive the biosynthetic reactions to completion.

Ultimately, at 440, the extracellular medium is separated from the microbial biomass (e.g., biomass produced in the bioreactor). At this element, the pigment compound has been produced extracellularly in the optimized medium and may be readily isolated and purified for use in suitable industrial applications such as textile, food and beverage, cosmetics, pharma, or other fields. The process 400 may facilitate efficient, scalable, and tunable production of pigment compounds, such as melanins, with reduced cellular toxicity and simplified downstream processing compared to traditional intracellular biosynthesis methods.

In accordance with another disclosed embodiment, the proposed engineered microbes (e.g. E. coli) optionally are inoculated onto soil (or relevant media such as roots, seeds, and plants), for example by spraying the microbial medium onto soil to overproduce melanin and help with carbon (e.g., from CO₂ in the atmosphere) capture, sequestration, and retention in soil. The interaction may help lock carbon in the soil for longer periods, potentially contributing to long-term carbon storage and mitigating climate change. Additionally, melanin's ability to absorb UV radiation and protect against oxidative stress may contribute to the preservation of other carbon compounds and organic matter in soil, further aiding in carbon sequestration. The sequestration of carbon in soil may help climate challenges and improve the soil nutritional resources, thereby improving plant growth.

In accordance with another aspect of the present disclosure, the properties of pigment polymers produced extracellularly may be modulated by adding to the extracellular medium, one or more capping or terminating agents. As used herein, “capping” or “terminating” agents refer to molecules that are capable of reacting with reactive sites (e.g., quinone, catechol, or indole moieties) on pigment monomers, oligomers (as used herein, ‘oligomers’ includes dimers, trimers, and higher-order short-chain polymers), or growing pigment polymers, thereby terminating further polymerization to the final pigment or colourant polymer. The addition of the terminating agent enables precise control over the average molecular weight, solubility, dispersibility, and color of the resulting pigment. The approach allows for the production of pigment polymers with tailored properties for specific industrial or material applications. The terminating agent may be introduced at any stage of the pigment production process, including during microbial fermentation, after fermentation, or in a separate in vitro element. The process may be performed in batch, fed-batch, or continuous mode, and is compatible with a wide range of microbial hosts and pigment biosynthetic pathways.

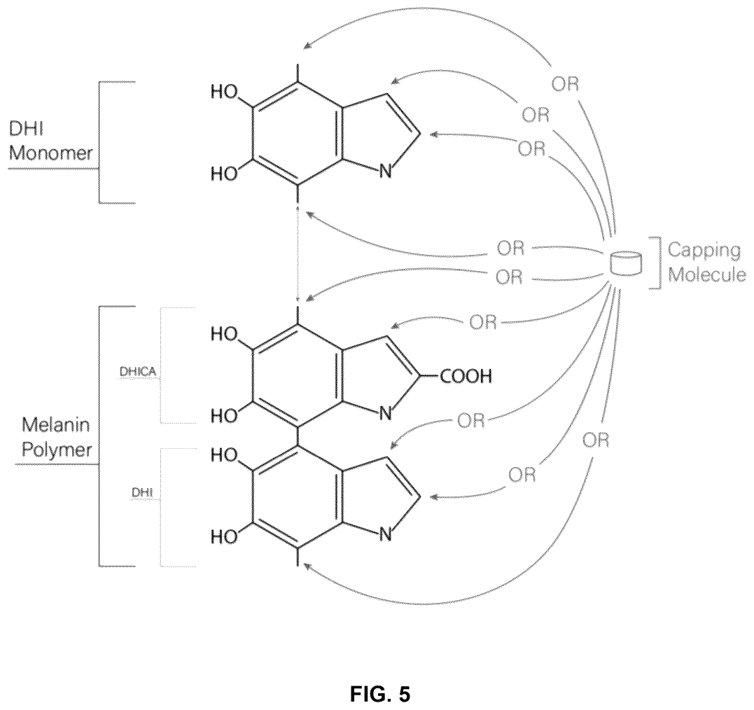

Referring to FIG. 5, a generic terminating agent is shown configured to interact with various reactive sites present on a growing colorant polymer (in this case melanin), as well as on oligomers and monomeric units thereof, such as DHI in the case of melanin biosynthesis. The terminating agent may bind to polymer extension sites along the polymer chain or to reactive groups on smaller molecular species, thereby blocking further polymerization. This mechanism enables precise control over the molecular weight, solubility, and other properties of the resulting pigment polymer by effectively capping the polymer at different stages of growth.

A wide variety of capping or terminating agents may be employed according to the present disclosure. Suitable agents include, but are not limited to, thiol-containing compounds (such as N-acetylcysteine, mercaptosuccinic acid, and thioglycolic acid), phenolic compounds (such as resorcinol), aromatic amines (such as p-aminophenol and tyramine), and other molecules capable of covalently or non-covalently interacting with reactive sites on pigment polymers. As used herein, a thiol-containing compound refers to a compound comprising a sulfhydryl (—SH) group. The selection of a particular capping or terminating agent, as well as the concentration, timing, and method of addition, may be selected and optimized to achieve a desired degree of polymerization, pigment molecular weight, solubility, color, or other physical or chemical property. In some embodiments, combinations of capping agents are used to further modulate pigment characteristics or to introduce functional groups for downstream applications, such as binding to substrates (e.g., textile or cosmetic substrates) or enhancing compatibility with formulation ingredients.

The concept of modulating pigment polymer properties by the use of capping or terminating agents is not limited to melanin or to pigments produced by microbial fermentation. The present disclosure encompasses the application of capping or terminating agents to any pigment polymer, or polymer in general, that forms via oxidative, enzymatic, or free-radical polymerization, regardless of the biological or chemical origin of the pigment (not limited to, poly-DOPA, poly-tyrosine, polyphenols, and other natural or synthetic pigment polymers. The process may be applied in vivo (e.g., during microbial or cellular biosynthesis), in vitro (e.g., in cell-free systems or after pigment extraction), or in hybrid systems combining biological and chemical elements. The ability to control pigment polymer properties through capping or terminating agents provides a versatile and generalizable platform for the production of pigments and functional polymers with customizable characteristics, supporting a broad range of current and future embodiments.

In certain embodiments, the capping or terminating agent is selected based on the reactivity with specific functional groups present on the pigment monomer or polymer. For example, thiol-containing agents are particularly effective at reacting with quinone intermediates formed during melanin biosynthesis, resulting in the formation of thioether linkages that block further polymer growth. The stoichiometry of capping agent to pigment monomer may be adjusted to achieve partial or complete termination, thereby tuning the average chain length and dispersity of the pigment polymer. The timing of capping agent addition may also be varied; early addition result in shorter, more soluble polymers, while later addition may allow for the formation of longer chains before termination.

The following examples are provided to further illustrate embodiments of the present disclosure relating to the extracellular production of colourants and modulation of pigment polymer properties using capping or terminating agents. These example are not intended to limit the scope of the invention.

### Example 1: Production of Melanin Using Engineered E. coli

To evaluate the ability of engineered E. coli strains to synthesize melanin from simple carbon and nitrogen sources, a series of strains with defined chromosomal mutations were constructed and tested for melanin production under minimal media conditions. The genotypes of the strains, as well as the presence or absence of relevant plasmids, are summarized in Table 1.

Each strain was grown in DMM Minimal medium supplemented with 1-5 g/L meat peptone, 0.1-1.5 mM CuSO₄, 0.1-2% glucose, 20-40 g/L ammonium sulfate, and 1 μg/mL each of tryptophan and phenylalanine to complement disrupted biosynthetic pathways. For each experiment, 30 mL of medium in a 250 mL flask was inoculated with 1 mL of an overnight culture grown in Lysogeny Broth (LB) medium. Cultures were incubated with shaking at 28 to 37° C. for 48-120 hours. After incubation, cells were removed by centrifugation and the supernatant was analyzed spectrophotometrically at 470 nm to detect the presence of melanin.

The results, shown in Table 1, indicate that melanin production was not detectable (N.D.) in wild-type strains or in strains lacking the tyrosinase expression plasmid. Introduction of a plasmid encoding Bacillus megaterium tyrosinase fused to a hemolysin signal peptide (pTase) enabled melanin production in strains with disruptions in aroP, tyrP, and tyrR. The highest levels of melanin were observed in strains including additional chromosomal disruptions (ptsl, zwf, trpE, pheA) and harboring the pTase plasmid, as indicated by the relative increase in melanin concentration (designated by plus signs). The presence of an additional plasmid-borne copy of yddG (pTaseY) further enhanced melanin production. In all strains, the YddG protein was supplied from the chromosomal yddG locus.

TABLE 1
Melanin Production by Engineered E. coli Strains

| | | Marker(s) | |
|---|---|---|---|
| Genotype | Plasmid | on plasmid(s) | Results |
| Wild type | | None | N.D. |
| Wild type | pTase | amp | N.D. |
| ΔaroP, ΔtyrP, ΔtyrR | | None | N.D. |
| ΔaroP, ΔtyrP, ΔtyrR | pTase | amp | + |
| ΔaroP, ΔtyrP, ΔtyrR | pTaseY | amp, kan, cam | +++ |
| ΔaroP, ΔtyrP, ΔtyrR, ΔptsI, | | None | N.D. |
| Δzwf, ΔtrpE, ΔpheA | | | |
| ΔaroP, ΔtyrP, ΔtyrR, ΔptsI, | pTase | amp | ++++ |
| Δzwf, ΔtrpE, ΔpheA | | | |

In Table 1, the genotype designations correspond to E. coli genes. pTase designates a plasmid harboring the B. megaterium tyrosinase fused to the hemolysin signal peptide. pTaseY designates a colE1-based plasmid expressing the same tyrosinase plus an additional copy of yddG. Amp=ampicillin, kan=kanamycin, cam=chloramphenicol. Plus signs indicate the presence and relative concentration of melanin in the media; N.D. indicates melanin was not detectable.

These results demonstrate that the combination of targeted chromosomal mutations and plasmid-borne expression of tyrosinase, particularly when combined with additional copies of yddG, enables efficient production of melanin from glucose and ammonium salts in minimal media. The level of melanin production is strongly influenced by the genetic background and the presence of specific biosynthetic and export elements.

### Example 2—Terminating Agents for Melanin

According to one embodiment, experimental results have demonstrated the effectiveness of modulating the melanin polymer using various capping agents. For example, in shake flask cultures of engineered E. coli producing melanin, the addition of N-acetylcysteine or mercaptosuccinic acid at various concentrations resulted in a marked reduction in the formation of high molecular weight, insoluble pigment. Instead, the majority of the pigment remained soluble in the culture supernatant, and the color of the broth may be modulated by the choice and amount of the capping agent. The transition point between complete inhibition and partial polymerization was observed by titrating the capping agent concentration, and the resulting pigment was characterized by visual inspection, solubility assays, and, where available, molecular weight analysis (e.g., gel permeation chromatography or mass spectrometry).

Engineered E. coli strains capable of producing melanin were cultivated in Lysogeny Broth medium supplemented with 0.5-3 g/L tyrosine. After autoclaving and cooling, copper (Cu²⁺) and iron (Fe³⁺) were added to a final concentration of 0.25-3 mM each. The medium was inoculated at 1% (v/v) from a fresh seed culture. After inoculation, the cultures were supplemented with one or more capping or terminating agents at varying concentrations. Capping agents tested included N-acetylcysteine, mercaptosuccinic acid, thioglycolic acid, resorcinol, and tyramine, each prepared as concentrated stock solutions. The agents were introduced either at the start of cultivation, during pigment formation, or after pigment accumulation, depending on the experimental design.

A matrix of capping agent concentrations was tested to determine the effect on pigment polymerization. The cultures were incubated under conditions favorable for pigment production. Aliquots of 0.1-0.5 mL of the inoculated medium were dispensed into wells of a deep-well 96-well plate. Capping agents were added at various concentrations, starting at a concentration indicated by A (in Table 2) which was selected at 40-70 mM and serially diluted two-fold across the plate. After the desired incubation period, the cultures were processed to separate the biomass from the extracellular medium. The solubility, colour, and apparent molecular weight of the pigment in the extracellular medium were assessed using standard analytical techniques. As a practical and rapid method for monitoring polymer molecular weight, the color of the cell pellet and the darkness of the culture broth were visually inspected in which a blackened cell pellet indicated the presence of high molecular weight, insoluble pigment, while a darker broth indicated a higher proportion of soluble, lower molecular weight pigment. The effect of each capping agent on pigment color was also monitored, as different agents may influence the hue or intensity of the resulting pigment. The results are summarized in the following Table 2.

TABLE 2
Experimental Results

| Terminating | Reagent | | Pellet |
|---|---|---|---|
| Agent | concentration (mM) | Broth Colour | Appearance |
| Mercaptosuccinic | A | Pale Yellow | No |
| acid | A/2 | Pale Yellow | No |
| | A/4 | Pale Yellow | Light brown |
| | A/8 | Light Brown | Light brown |
| | A/16 | Medium Brown | Dark Brown |
| Tyramine | A | Colorless | No |
| | A/2 | Pale Yellow | No |
| | A/4 | Pale Yellow | No |
| | A/8 | Light Brown | Light Brown |
| | A/16 | Light Brown | Light Brown |
| Resorcinol | A | Colorless | No |
| | A/2 | Pale Yellow | No |
| | A/4 | Dark Brown | Dark Brown |
| | A/8 | Medium Brown | Dark Brown |
| | A/16 | Light Brown | Dark Brown |
| Thioglycolic acid | A | Colorless | No |
| | A/2 | Colorless | No |
| | A/4 | Colorless | No |
| | A/8 | Pale Yellow | No |
| | A/16 | Medium Brown | Medium Brown |
| N-Acetyl-L- | A | Pale Yellow | No |
| cysteine | A/2 | Pale Yellow | Light brown |
| | A/4 | Light Brown | Light brown |
| | A/8 | Medium Brown | Medium Brown |
| | A/16 | Medium Brown | Medium Brown |

### Example 3: Modulation of Soluble Melanin Yield by Thioglycolic Acid Addition

In a separate experiment, the effect of thioglycolic acid concentration on the production of soluble melanin was evaluated. Engineered E. coli strains producing melanin were cultivated in LB medium under conditions similar to those described in Example 1. Thioglycolic acid was added to the cultures at various concentration increments indicated by C in Table 3, and the amount of soluble melanin in the supernatant was measured after incubation.

The results, summarized in Table 3, demonstrate that the addition of thioglycolic acid at certain concentrations increased the yield of soluble melanin. However, addition of thioglycolic acid beyond an optimal concentration resulted in decreased melanin synthesis, likely due to excessive blocking of monomers and small oligomers before they were able to polymerize into larger, more intensely colored molecules.

TABLE 3
Effect of Thioglycolic Acid Concentration
on Soluble Melanin Yield

| | Thioglycolic acid concentration (nM) | | | | | |
|---|---|---|---|---|---|---|
| | 0 | C | 2C | 4C | 6C | 8C |
| Soluble Melanin (g/l) | 0.28 | 0.33 | 0.31 | 0.49 | 0.52 | 0.36 |

These results further illustrate that the concentration of a terminating agent can be optimized to maximize the production of soluble pigment, and that excessive terminating agent may inhibit overall pigment synthesis by prematurely capping reactive intermediates

The present invention further contemplates the use of capping or terminating agents to introduce additional functionality to the pigment polymer. For example, capping agents bearing reactive handles, affinity tags, or chromophores are used to impart new properties to the pigment, such as enhanced binding to textile fibers, improved compatibility with cosmetic formulations, or altered optical characteristics. The process is also compatible with the use of chain-terminating analogs of pigment monomers, which may be incorporated into the growing polymer to further modulate its structure and properties.

Advantages of the proposed biosynthesis system and processes include:
a) Some pigment molecules or compounds, such as Melanins, are large polymers and are likely to precipitate once synthesized. Such pigment molecules, if produced intracellularly, may be toxic to cells since they bind to surfaces inside the cell, thereby impairing cell viability and limiting the overall yield of the pigment molecule and productivity of the cell. Production of such pigment molecules extracellularly may eliminate this toxicity and result in higher yields and productivity.
b) Productivity of pigment molecules is not limited by biomass of the cells. Thereby, the overall productivity does not solely rely on maximizing biomass, and for example, allow more nutrition (e.g. carbon and nitrogen) to feed into precursors (e.g. tyrosine) and pigment (e.g. melanin) production.
c) Engineered cells may be optimized for productivity per unit time, thereby improving efficiency of processes over other extracellular melanin production systems.
d) The presented approach for melanin production exports tyrosinase and manipulates expression of tyrosine transporters to obtain a net flow out of the cell and into the growth media or fermentation broth during the growth process.
e) Production of pigment molecules (e.g. melanin) extracellularly, may typically result in simpler harvest and purification of the pigment from the growth media or fermentation broth, compared to intracellular pigment production methods.
f) The proposed process enhances pigment yields by inherently keeping cells healthy and productive, permitting continual production of pigments (e.g. melanin).
g) The proposed process allows producing melanins of different colours by modifying the availability of different monomers for polymerization into melanin.
h) The proposed processes may capture carbon as melanin, which is an important molecule for storing carbon in soils. Microbes engineered to overproduce melanin may be applied to soils, for example by spraying, to increase the amount of soil carbon sequestered, thereby reducing atmospheric carbon.
i) The disclosed process enables the selective and economical production of pheomelanin by tuning the ratio of cysteine to dopaquinone, overcoming the prior art limitation of uncontrollable copolymer colour and composition.
j) The present disclosure provides enablement for precise control over the molecular weight, solubility, and color of pigment polymers by the addition of capping or terminating agents, such as thiol-containing compounds, to the extracellular medium. The terminating agents react with reactive sites on pigment monomers, oligomers, or growing polymers, thereby blocking further polymerization and allowing the production of pigment polymers with tailored properties for specific industrial, cosmetic, or material applications.
k) The use of terminating agents in the pigment polymerization process is broadly applicable to a variety of pigment systems, including but not limited to melanins, and may be implemented during or after microbial fermentation, or in in vitro polymerization reactions.

While specific embodiments have been described and illustrated, such embodiments should be considered illustrative only and not as limiting the disclosed embodiments as construed in accordance with the accompanying claims.

1. A process for extracellular production of a colourant compound, the process comprising:
producing a first intermediate by an engineered microbial cell and exporting the first intermediate to an extracellular medium;
producing a second intermediate by the microbial cell and exporting the second intermediate to the extracellular medium, wherein the second intermediate is active upon export; and
reacting the first and second intermediates in the extracellular medium to produce the colourant compound.

2. The process of claim 1, wherein the first intermediate is an amino acid and the second intermediate is an enzyme.

3. The process of claim 2, wherein the second intermediate is poorly active or inactive within the cell and more active after export.

4. The process of claim 1, wherein the cell is engineered to overexpress or upregulate one or more genes encoding exporters of the first intermediate, and to disrupt, delete, or downregulate one or more genes encoding importers of the first intermediate to prevent reuptake of the first intermediate.

5. The process of claim 1, wherein the second intermediate is fused to a signal peptide that facilitates export to the extracellular medium.

6. The process of claim 1, wherein the colourant compound is a melanin molecule, the first intermediate is tyrosine, and the second intermediate is a tyrosinase.

7. The process of claim 1, wherein the colourant compound is a violacein molecule, the first intermediate is tryptophan, and the second intermediate is a tryptophanase.

8. The process of claim 6, wherein the cell is engineered to disrupt, delete, or downregulate the tyrP and aroP genes and to overexpress or upregulate the yddG gene.

9. The process of claim 1, further comprising producing a third intermediate by the cell and exporting the third intermediate outside the cell, wherein the third intermediate reacts with the first and the second intermediates to produce the colourant compound.

10. The process of claim 9, wherein the third intermediate comprises an enzyme or a compound capable of altering or modulating the composition, color, or properties of the colourant compound by reacting with the first and/or second intermediates.

11. The process of claim 10, wherein the third intermediate is selected from the group consisting of DOPAchrome tautomerase, DHICA oxidase, cysteine, and glutathione.

12. The process of claim 11, wherein the cell is engineered to overproduce and export cysteine and/or glutathione as the third intermediate by overexpressing a cysteine exporter and disrupting, deleting, or downregulating one or more genes encoding cysteine importers.

13. The process of claim 1, wherein the process further comprises cultivating the cell in a growth medium, a fermentation medium, or broth, and wherein the process is performed by batch, fed-batch, or continuous fermentation.

14. The process of claim 13, wherein the cultivation or fermentation is conducted under anaerobic conditions to minimize polymerization of the colourant compound and to accumulate intermediates of the colourant compound.

15. The process of claim 14, wherein the intermediates are separated from a biomass resulting from the culture growth for later use in other chemical processes including production of the colourant compound.

16. The process of claim 1 further comprising adding one or more terminating agents to the extracellular medium to modulate the molecular weight, solubility, or color of the colourant compound.

17. The process of claim 16, wherein the one or more terminating agents are selected from the group consisting of thiol-containing compounds, phenolic compounds, and aromatic amines.

18. The process of claim 16, wherein the one or more terminating agents are added during fermentation and/or after fermentation, wherein the amount and timing of the addition of the one or more terminating agents is selected to achieve a desired average molecular weight or solubility of the colourant compound such that the resulting colourant compound has a controlled molecular weight and is soluble in aqueous solution.

19. The processes of claim 1, further comprising introducing the microbial cell into an outdoor environment, including soil, roots, seeds, or plants, wherein the microbial cell produces and/or facilitates the accumulation of pigment compounds that increase the concentration of fixed carbon in the environment.

20. A genetically engineered microbial cell for improved production of a pigment compound relative to a parental strain, wherein the pigment compound is produced outside the microbial cell by reaction of a first intermediate and a second intermediate, the microbial cell configured to:
produce the first intermediate and export the produced first intermediate outside the cell; and
produce the second intermediate and export the produced second intermediate outside the cell, wherein the second intermediate is poorly active or inactive within the cell and more active after export.
